(19) United States
(12) Patent Application Publication
(10) Pub. No.: US 2008/0286357 A1
(43) Pub. Date: Nov. 20, 2008

(54) MULTI-FUNCTIONAL PARTICULATE DELIVERY SYSTEM FOR PHARMACOLOGICALLY ACTIVE INGREDIENTS

(75) Inventors: Bob Sherwood, Wood, SD (US); Janos Szamosi, Greentown, PA (US); Paul Richardson, Vernon, NJ (US)

Correspondence Address: HOFFMANN & BARON, LLP, 6900 JERICHO TURNPIKE, SYOSSET, NY, 11791, US

(73) Assignee: Balchem Corporation, New Hampton, NY (US)

(21) Appl. No.: 11/750,078

(22) Filed: May 17, 2007

(51) Int. Cl. A61K 9/26; A61K 31/16; A61K 33/00; A61K 33/04; A61K 33/10; A61K 33/42; A61K 9/14

(52) U.S. Cl. 424/468; 424/484; 424/488; 424/602; 424/686; 424/687; 424/709; 424/711; 424/715; 424/94.1; 514/629

(57) ABSTRACT

The present invention relates to a pharmacologic dosage unit which includes a multi-functional particulate system and a pharmacologically active agent. The multi-functional particulate system includes solid biologically-safe particles incorporated into a matrix and having a characteristic that each individual particle retains its original size. The invention also relates to methods of preparing, enhancing flow properties and compacting properties of a pharmacologic composition, increasing capacity for inclusion of ingredients, and increasing dispersion of actives. Finally, the invention includes a method for delivering an active agent to a patient.

### BACKGROUND OF THE INVENTION

The present invention relates to delivering pharmacologically active ingredients, and, in particular, to delivery systems having improved processability and drug delivery characteristics.

Compressed tablets are well known in the art of delivering pharmacologically active ingredients. They provide a convenient and efficient vehicle for delivering uniform doses of pharmacologically active ingredients. However, there are several divergent technical requirements which must be met in order to implement manufacture of pharmacologically active dosage units in the form of compressed tablets.

For example, in order to ensure accurate and uniform delivery of the required amount of active ingredient, a substantially homogeneous blend of different tablet ingredients must be prepared and maintained throughout the tableting process. This requirement can be quite difficult to achieve, especially when the active ingredient(s) resists blending. Thus, pharmacologically active ingredients which are, inter alia, fine, cohesive, etc., such as acetaminophen, can present very difficult tableting problems resulting in non-uniform doses and/or inferior tablets.

Furthermore and closely related to maintaining uniformity of mixture, a tableting mixture must have a good flow characteristic. This means that the mixture is able to flow through the machinery which delivers it to the tableting press in such a way that that the mixture remains uniform and flowable without substantial discontinuity.

Another characteristic required of the tableting mixture is that h demonstrates good compactability at reasonable compression forces. To a certain degree maintaining this characteristic can he inimical to maintaining a thorough and homogenous mixture with good flowability. Basically, highly flowable, thoroughly-mixed ingredients many times resist compaction under reasonable compression forces.

As a result of requirements such as those set forth above and others, tablet manufacturers are required to call upon many and sometimes expensive ingredients to meet the particular deficiencies of specific tableting mixtures. Furthermore, expensive tableting equipment and/or modifications thereof can be required for processing different tableting mixtures.

Finally, since delivery of certain active ingredients require different types of release profiles, even further modifications and/or ingredients must be made to mixtures and equipment and processing parameters in order to achieve the desired release in the host patient. Consequently those skilled in the art continuously search for new and better methods and ingredients to enhance tablet mixing and tablet formation to engineer the correct compressed tablet for each application.

Therefore, it is the purpose in the present invention to provide a new and unexpectedly effective composition and method for processing and making dosage units for delivering a pharmacologically active ingredient.

### SUMMARY OF THE INVENTION

The present invention is a pharmacologic dosage unit which includes a pharmacologically active agent and a multi-functional particulate system. The present invention also includes methods of preparing and making a pharmacologic dosage unit. Other aspects of the invention involve processes for enhancing flow properties and compacting properties of a pharmacological composition used as an ingredient for a pharmacologic dosage unit. Additionally, the invention involves a method for delivering a pharmacologically active agent to a patient and a method for increasing capacity for inclusion of at least one ingredient in a pharmacologic dosage unit.

Dosage units of the present invention include, but are not limited to, tablets, sachets, lozenges, hard capsules, softgels, troches, dragees, suppositories, and the like. A preferred dosage unit is a compressed tablet.

The dosage units contain a pharmacologically active agent and a multi-functional particulate system. The multi-functional particulate system includes, (i) solid biologically-safe particles, (ii) incorporated into a matrix, (iii) and having a characteristic that each individual particle retains its original size and the matrix, being present in a weight ratio of from 1:99 to 99:1, and (iv) such that the mean weight diameter of resulting particulates have a size of from 25 to 500 microns.

The matrix is a biologically-safe material that can be selected from the group consisting of polysaccharides, modified polysaccharides, sugars, gums, thickeners, stabilizers, syrups, sugar alcohols, flours, starches, dextrose, maltodextrins, cellulose, and combinations thereof. Preferably, the matrix material is sugar or mixtures of sugars.

In a preferred embodiment, the multi-functional particulate system displays a loose bulk density of from about 0.1 to about 1.1 kg/L. In another preferred embodiment, the mean weight diameter of the multi-functional particulate system is from 50 to 400 microns. Preferably, the weight ratio between the particles and the matrix ranges from 80:20 to 20:80, and more preferably 40:60 to 60:40. The particulate has discrete particles with a size in the range of from 2 to 275 microns. The particles can be nutritionally active.

The particles which are used in the multi-functional particulate system are biologically-safe and can be inorganic substances such as Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, and Ca(HPO₄), and Ca₃(PO₄)₂, or organic substances such as celluloses, polysaccharides, and polymerics. The particles can also be biologically-safe acids such as citric acid, maleic acid, tartaric acid, maleic acid, lactic acid, acetic acid, and combination thereof, and derivatives and salts thereof. In a particularly preferred embodiment, the particle is CaCO₃.

Furthermore, the dosage unit of the present invention can be used for pharmacologically active agents such as those selected from the group consisting of antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, and-ulcer agents, and-inflammatory substances, coronary dilators, cerebral psychotropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

In a particularly preferred embodiment, the pharmacologically active agent can be acetaminophen. Another pharmacologically active agent can be the cofactor Coenzyme Q10(CoQ10).

In another embodiment, the invention relates to a method of preparing a pharmacologic dosage unit. The method involves admixing a multi-functional particulate system and a pharmacologically active agent in amounts requisite to provide a pharmacologic composition capable of use as an ingredient for inclusion in a pharmacologic dosage unit. The pharmacologic composition has enhanced flowability and enhanced comparability. These features are particularly valuable when the dosage unit being prepared is a compressed tablet.

In yet another embodiment, the invention relates to a method for making a pharmacologic dosage unit comprising (i) admixing a multi-functional particulate system and a pharmacologically active agent in amounts requisite to provide a pharmacologic composition which is sufficiently flowable and compactable to form a compressed tablet, and (ii) compacting the mixture resulting from step (i) sufficiently to form a compressed tablet.

In another embodiment, the invention relates to a process for enhancing the flow properties of a pharmacologic composition used as an ingredient for a pharmacologic dosage unit. The process involves adding a multi-functional particulate system to at least one pharmaceutically active agent in an amount sufficient to improve flowability of the active agent in its use as an ingredient for making a pharmacologic dosage unit.

In yet another embodiment, the invention relates to a process for enhancing compacting properties of a pharmacologic composition used as an ingredient for a pharmacologic dosage unit. The process involves adding a multi-functional particulate system to at least one pharmaceutically active agent in an amount sufficient to provide or improve compactability of the active agent whereby it can be used as an ingredient for making a pharmacologic dosage unit.

In another embodiment the invention relates to a method for delivering a pharmacologically active agent to a patient. The method involves administering to a patient, in need of treatment with at least one pharmacologically-active agent, a pharmacologic dosage unit, containing (i) at least one pharmacologically active agent suitable for treatment of the patient and (ii) a multi-functional particulate system.

In yet another embodiment, the invention relates to a method for increasing capacity for inclusion of at least one ingredient in a pharmacologic dosage unit. The method involves adding a multi-functional particulate system to a pharmacologic composition used in a pharmacologic dosage unit in an amount sufficient to increase the amount of at least one other ingredient in the composition. The pharmacologic composition may contain silicified microcrystalline cellulose, vinyl-pyrollidone vinyl-acetate copolymer, or combinations thereof

In another embodiment, the invention related to a method for increasing dispersion of pharmacologically active agents within a pharmacologic dosage unit. The method involves adding a multi-functional particulate system to at least one pharmacologically active agent in an amount sufficient to provide or improve dispersion of said pharmacologically active agent whereby its inclusion as an ingredient in a pharmacologic dosage unit is facilitated.

For a better understanding of the present invention, together with other and further advantages, reference is made to the following detailed description, and its scope will he pointed out in the claims.

### DETAILED DESCRIPTION OF THE INVENTION

The present invention provides a pharmacologic dosage unit which includes a pharmacologically active agent and a multi-functional particulate system.

The pharmacologic dosage unit can be in forms that are delivered orally or rectally and may include, but are not limited to, tablets, caplets, minitablets, sachets, lozenges, hard capsules, softgels, troches, dragees, sublingual tablets, buccal tablets, suppositories, and the like. The preferred dosage units are compressed tablets and capsules.

### Compressed Tablets

Compressed tablets can be made according to various methods well known in the art. For example, see Rudnic, E., and Schwartz, J. B. Oral Solid Dosage Forms, Chapter 92, Tablets, pp. 1615-1641, in Remington's, 19th Ed.

Compressed tablets can be administered to a patient by numerous means. For example, the compressed tablets may be swallowed or chewed. The application may be sublingual, buccal, enteric, immediate release, controlled release, rapid dissolve, modified release delayed release, or pulsatile release.

The compressed tablets of the present invention contain pharmacologically active agents and a multi-functional particulate system. The tablets may also contain other ingredients such as additives that serve a variety of different functions.

### Pharmacologically Active Agent

The pharmacologically active agent, can be a prescription drug, a non-prescription drug, or a vitamin. Examples of pharmacologically active agents useful in the present invention include antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, anti diarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof. Preferably, the pharmacologically active agent is acetaminophen, CoEnzyme Q10, or B-vitamins.

The pharmacologically active agent can also be encapsulated. For example, the active agent may be encapsulated in a polymer and/or other coating materials such as lipids. Polymer and lipids for use as protective coatings of an active agent are known to those skilled in the art. Lipids usually are fat and fat-like substances which are derived either synthetically or from plants or animals.

### Multi-Functional Particulate System

The multi-functional particulate system contains solid biologically-safe particles incorporated into a matrix. The particles are incorporated in a manner in which each individual particle retains its original size.

The mean weight diameter as measured using sieve analysis of the resulting particulate system ranges from 25 to 500 microns. Preferably the mean weight diameter ranges from 2 to 275, more preferably from 5 to 250 microns, and most preferably from 7 to 200 microns.

The matrix can be selected from abroad range of materials as long as they are biologically-safe. Preferably, the matrix is selected from the following: polysaccharides, modified polysaccharides, sugars, gums, thickeners, stabilizers, syrups, sugar alcohols, flours, starches, dextrose, maltodextrins, and celluloses. Most preferably, the matrix is composed of a sugar or mixtures of sugars. The particle size of the matrix materials can vary between 1 and 400 microns, preferably between 5 and 200 microns, more preferably between 25 and 100 microns.

Preferably, the multi-functional particulate system displays a loose bulk density of 0.1 to 1.1, more preferably 0.3 to 0.6 kg/L. Loose bulk density is measured by measuring the volume of a known mass powder sample, that has passed through a screen into a gradulated cylinder. The procedure is described in USP<616<Bulk Density and Tapped Density.

The biologically-safe particles are in the matrix in a weight ratio of 1:99 to 99:1. Preferably, the weight ratio is 20:80 to 80:20, and most preferably from 40:60 to 60:40.

The particles can be of very different nature. In particular, nutritionally active particles that improve the oral properties of the dosage unit, or the bioavailability of the particle or the dispensability of the particle in a dosage unit have proven to be very useful. Preferred particles have low water solubility.

Examples of inorganic particles are one or more of the particles selected from the group consisting of: alkali or alkali earth metals such as salts of Na, K, Ca and Mg, in particular the carbonates, sulfates, and phosphates, Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, Ca(HPO₄), and Ca₃(PO₄)₂. While examples of organic particles include organic acids such as citric acid, maleic acid, tartaric acid, maleic acid, lactic acid, acetic acid or derivatives or salts thereof, and microcrystalline cellulose, silicified microcrystalline cellulose, hydroxypropylcellulose, hydroxy propylmethylcellulose, lactose, dextrose, cyclodextrin, polyvinylpyrrolidone, polyvinylpyrrolidone vinyl acetate), polyacrylamides, starches, pregelatinize starches, gelatin, methyl cellulose, sodium carboxymethylcellulose, ethyl cellulose, polyvinyloxoazolidone, and polyvinyalcohols. Preferably, the particles are CaCO₃.

The discrete particle size of the biologically-safe particle in the total particulate system suitably ranges from of 2 to 275 microns. Preferably the particle size is 5 to 250, and most preferably 7 to 200 microns.

### Optional Additives

As indicated above, the present solid dosage form may optionally comprise a further additive typically selected from a disintegrating agent, binder, lubricant, flavoring agent, preservative, colorant, and any suitable mixture thereof.

Disintegrating agents enhance the rate of which the tabletted multi-functional particulate system disintegrates. Examples of disintegrating agents include, but are not limited to, crosscarmellose sodium, sodium starch glycolate, cross-linked polyvinylpyrollidone, soy polysaccharides, pregelatinized starches, calcium silicate, sugar combinations, or combinations thereof or agents which increase salivation in the oral cavity such as organic acids, including, but not limited to, citric, maleic, or tartaric acid and their encapsulated, or otherwise modified forms.

Other ingredients which can be included in the dosage unit of the present invention include binders. Binders generally contribute to the ease of formation and general quality of the dosage units. Non-limiting examples of binders include starches, pregelatinized starches, gelatin, polyvinylpyrrolidone, methyl cellulose, hydroxypropylcellulose, hydroxypropylmethylcellulose, sodium carboxymethylcellulose, ethylcellulose, polyacrylamides, polyvinyloxoazolidone, and polyvinyalcohols.

### Enhanced Flowability

Flowability of an ingredient such as a pharmacological composition for making a dosage unit can be defined as the property of the composition, e.g., powder or granulate, which enables it to flow through processing machinery and ultimately into a dosage-forming station. Poorly flowing compositions such as powders present many difficulties in the pharmaceutical industry in processing and dosage-forming, especially in the making of compressed tablets and filled capsules. It is, indeed, desirable to provide a free flowing composition, e.g., a powder, so that certain advantages can be realized such as those set forth below:

(a) the composition easily flows into and through a machinery which passes it along for dosage-forming;

(b) as a consequence of uniform tablet weight and uniform doses of active ingredient, other parameters of finished tablets such as hardness, friability, disintegration characteristics, dissolution and plasma levels in the body are reproducible;

(c) high uniformity of the compressing composition or powder and, therefore, less wear on the machine;

(d) ease of ejection of air during compression because of good permeability and, therefore, fewer defective tablets due to capping or splitting; and

(e) high production rate allowed by the easily-flowing rate of a free flowing composition.

Many efforts have been made to overcome problems created by poor-flow properties by studying different formulations and applying different manufacturing techniques. At the same time many attempts have been made to establish experimental procedures having practical industrial significance which can measure or assess the intrinsic flowability of the powder to be encapsulated or compressed. A number of authors identified the powder flowability with the interparticulate friction, of which an “angle of repose” is a manifestation (E. Nelson—J. Am. Pharm. Assoc. Sci. Ed. 44, No. 7, 435-437 (1955)) and at least four practical methods of measuring the angle of repose have been developed which are described and compared by David Train in J. Pharm. Pharmacol. 10, 127T to 135T (1958).

Basically, according to the “angle of repose” method the powdered material is allowed to fall freely through an orifice onto a flat surface to form a conical pile of deposited material and the angle between the surface of the cone and the horizontal plane is known as the angle of repose. A high angle would indicate a poorly flowing material whereas a low angle would indicate good flow.

Timed delivery through an orifice is another method often used for evaluating the flowability of materials. A timer, e.g., a stop watch, is used to either time certain weights of powder flowing through the orifice or to close the orifice after a given time so that the powder flowing through in a designated time period can he weighed.

Ausburger and Shangraw (J. Pharm. Sci., 55, No. 4, 418-423 (1966)), attempted to evaluate and compare the free flowing properties of powders using the weight and weight variation of the finished tablets as the measured parameter. It was believed, in fact, that the weight variation of both capsules and tablets is directly dependent upon the reproducibility of powder flow into a fixed volume receptable (which may be a tablet die cavity or a capsule shell) and that good precision, which reflects in a high tablet weight and lower coefficient of variation, can only be obtained when the powder to be filled has a good flowability. This method can be conveniently used in routine quality control tests.

A further method for determining the free flow properties of a powder has been described in DEGUSSA Schriftenreihe, Anwendungstechnik Pigmente Nr. 31 (Wolfgang Hanau (Main)) pages 6 to 8 and is based on how the powder runs through sand timer-like funnels with varying orifices. The equipment there described consists of a series of five glass funnels with orifice diameters of 2.5, 5, 8, 12, and 18 mm, and the powder flowability in ranked as outstanding, very good, good, acceptable or poor depending on the diameter of the orifice which the powder can still pass through.

And, in U.S. Pat. No. 4,274,286, an apparatus for measuring the flowability of powder is disclosed which utilizes a container having a bottom wail with an opening therein. The bottom wall may be changed to provide openings of different sizes. The test consists of allowing the powder to flow through successively smaller holes. The smallest hole size through which the powder will freely flow is a measure of its flowability.

In any event, by use of one or more of the methods set forth above, pharmacologic compositions prepared in accordance with the present invention have been found to exhibit greater flowability than, at least, the active component by itself and without the inclusion of the multi-functional particulate. In fact, active ingredients such as CoQ10 exhibit virtually no flowability in the absence of the multi-functional particulate of a present invention. Moreover, the use of the multi-functional particulate and pharmacologic compositions resulting therefrom also enables (or improves) ingredients other than actives also be useable in dosage unit production machines in the pharmaceutical industry. These enhanced flowability characteristics are especially useful in making compressed tablets and in filling capsules.

### Enhancing Compactability and Dispersion of Actives

The present invention also includes the aspect of improving the comparability of a pharmacological composition which includes a pharmacologically active agent. By use of a multi-functional particulate of the present invention, a composition can be prepared, e.g., powder and/or particulate, which improves its compactability when compared to compactability of the active ingredient itself. In recent years pharmacologically active ingredients are routinely used in lower amounts per unit dosage than ever before and require the highest degree of accuracy possible. In order to implement these requirements, the additional ingredients must be capable of homogenously diluting the active ingredient and provide a sufficient reduction in the amount of elasticity and cohesivity to predictably separate and isolate mechanically grains of active ingredient.

Likewise, cohesive pharmacologically active ingredients cause problems during processing of dosage units because cohesive active ingredients clump together and remain substantially indispersable. Indispersable actives leads to the inability to create a uniform dosage unit. By use of the multifunctional particulate of the present invention, islands or “clumps” of active are virtually eliminated.

Consequently, the present invention provides a pharmacologic composition which is useful in preparing direct compression tablets, and low compression pre-formed inserts for use in capsule type unit dosage forms. Pre-forms can be prepared by “tamping”) rather than direct high pressure compression.

### Enhancing Carrying Capacity

The multi-functional particulate delivery system of the present invention also increases the capacity for inclusion of at least one ingredient in a pharmacologic dosage unit. For example, acetaminophen can be incorporated into the multi-functional particulate at higher percentage levels and successfully tabletted than compared to a formulation without the multi-function particulate.

Additionally, excipients can be added to the multi-functional particulate to further increase the carrying capacity of at least one ingredient. Examples of useful excipients include silicified microcrystalline cellulose, vinyl-pyrollidone vinyl-acetate copolymer, or combinations thereof. ProSolv/S630 is seven-to-one blend of silicified microcrystalline cellulose and vinyl-pyrollidone vinyl-acetate copolymer.

In the examples section, the carrying capacity of the multi-functional particulate delivery system is measured. The carrying capacity refers to the amount of a specific ingredient which can be incorporated into a particular dosage unit.

Methods of measuring carrying capacity are well known to practitioners in the art. For example, the carrying capacity of a tablet, is measured by taking into account the hardness range of the tablet, the amount of lbs of compression required to form the tablet, and the amount of lbs required to eject the tablet from the tableting machinery.

### Method of Making

The invention also relates to a method of preparing a pharmacologic dosage unit. The dosage unit is made by admixing a multi-functional particulate system and a pharmacologically active agent in amounts requisite to provide a pharmacologic composition capable of use as an ingredient for inclusion in a pharmacologic dosage unit.

In another embodiment, the invention relates to a method of making a pharmacologic dosage unit by admixing a multi-functional particulate system and a pharmacologically active agent in amounts requisite to provide a pharmacologic composition which is sufficiently flowable and compactable to form a compressed tablet, and compacting the resultant mixture sufficiently to form a compressed tablet.

Multi-functional particulate systems can be made by methods known in the art. A description of making a multi-functional particulate can be found in U.S. Pat. No. 6,673,383which is incorporated herein by reference.

Other processes typically used for powder granulations can also be used to produce the multi-functional particulate system, such as low and high shear mixer granulators.

For example, the multi-functional particulate system can be made using a fluid-bed drier top spray. The solid biologically-safe particles and a portion of the matrix components are combined. The remaining matrix components, preferably maltodextrin and dextrose, are mixed together with warm water at 25° C. to form a spray solution.

The outlet temperature and air volume are set to fluidize the particle/matrix mixture. Then, the spray solution is sprayed onto the particle/matrix mixture. The optimal spray rate and atomization air pressure can be determined without undue experimentation. For instance, a spray rate of 60 grams per minute with an atomization air pressure of 2.8 bar can be used.

After the mixture is completely sprayed, the multi-functional particulate is dried. The determination of optimal outlet air temperature and length of drying are within the purview of a person skilled in the art. For example, the particulate can be dried at an outlet air temperature of 40° C. for 5 minutes.

The multi-functional particulate can then be measured for loss on drying. A Metier LP-16 Moisture Balance can be used for this determination.

The last step involves passing the particulate through a sifter to remove oversized materials. For instance, a US#20 mesh and Sweco Sifter can be used.

Admixing a multi-functional particulate system to a pharmacologically active agent can be accomplished by various methods known in the art. Tumble mixers are often employed.

For example, a multi-functional particulate system and a pharmacologically active agent can be added to a V-blender, then mixed for an appropriate period of time at a given speed. The length of time and speed of rotation is subject to such factors as the active agent employed, the amount of multi-functional particulate, and the multi-functional particulate employed. Such variations are within the capabilities of those skilled in the art without the need for undue experimentation.

The invention also involves a method of making a pharmacologic dosage unit. The method involves admixing a multi-functional particulate system with a pharmacologically active agent in an amount sufficient to provide a flowable and compactable compressed tablet ingredient or mixture, and compacting the mixture to form a compressed tablet which can be recovered from compaction and stored and made available to a patient as a dosage unit.

Methods of compacting mixtures to form a compressed tablet are well known. A person skilled in the art would be able to determine, without the need for undue experimentation, the appropriate amounts of pressure to apply to the mixture, the optimal amounts of multi-functional particulate to include in the mixture, and the additives to include in order to create a tablet that could be recovered from compaction and stored.

The features and advantages of the present invention are more fully shown by the following examples, which are provided for purposes of illustration, and are not to be construed as limiting the invention in any way.

### EXAMPLES

### Example 1

### Process for Making a Multi-Functional Particulate System

Equipment: A pilot-scale 5 kg fluid-bed drier top spray

### Formulation:

$$\frac{\begin{matrix}2.5 kg 6 \times sugar ( 50 \% 6 \times sugar ) \\ 1.325 kg calcium carbonate ( 26.5 \% CalEssence 300 ) \\ 1.115 kg dextrose ( 22.3 \% Clintose Dextrose ) \\ 0.03 kg maltodextrin ( 0.6 \% Maltrin M \text{-} 100 ) \\ 0.03 kg microcrystalline cellulose ( 0.6 \% Avicel PH 101 )\end{matrix}}{5.00 kg TOTAL WEIGHT}$$

### Protocol:

The 6×powder sugar, calcium carbonate, 1.085 kg of dextrose, and macrocrystalline cellulose were placed into a bowl. The maltodextrin and the remaining 0.03 kg of dextrose were mixed in warm water at 25° C. (1.0% solids in solution) as the spray solution.

An outlet temperature range of about 38° C. was used and an air volume sufficient (damper ½ open) to fluidize the product was set. The spray solution was sprayed onto the product in the bowl at a spray rate of 60 grams per minute with an atomization air pressure of 2.8bar.

Upon completion of the spray solution, the multi-functional particulate system was dried at an outlet air temperature of 40° C. for 5 minutes. The multi-functional particulate system was sized on a US#20 mesh using a Sweco Sifter to remove any oversized material.

The multi-functional particulate system described above was used in the formulations below.

### Example 2

### Compaction of the Multi-Functional Particulate System

Tablets of various weights (between 1160 and 2600 mg) containing only the multi-functional particulate system were made. The compression profile for the 2000 mg tablets was measured.

Equipment: V-blender; instrumented Stokes RB-2 press; four 11/16″ flat-faced beveled-edged punches.

### Formulation:

$$\frac{\begin{matrix}1761.0 g multi \text{-} functional particulate system ( \sim 99 \% ) \\ 18.0 g magnesium stearate ( \sim 1 \% )\end{matrix}}{1779.0 g TOTAL WEIGHT}$$

### Results:

TABLE 1
Three sets of tablets made at different weights, at roughly the
same compression

| | Tablet | Thickness | Hardness range | Ejection |
|---|---|---|---|---|
| Compression (lbs) | Weight (mg) | (mm) | (kP) | (lbs) |
| 5200 | 1160 | 3.38 | 5.8-6.3 | 30 |
| 6200 | 1460 | 4.12 | 8.5-9.3 | 51 |
| 5100 | 2600 | 7.32 | 14.0-14.2 | 45 |

All three formulations tabletted without any problem. The multi-functional particulate system by itself can be tabletted at reasonable compression forces to create tablets within a good hardness range.

TABLE 2
The fourth set of tablets was made at 2000 mg tablet weight, at
different compressions

| | Tablet | Thickness | Hardness range | Ejection |
|---|---|---|---|---|
| Compression (lbs) | Weight (mg) | (mm) | (kP) | (lbs) |
| 1400 | 2050 | 6.82 | 1.9-2.1 | 0 |
| 1980 | 2000 | 6.56 | 3.3-3.8 | 4 |
| 2900 | 2040 | 6.27 | 5.2-5.8 | 12 |
| 3600 | 2060 | 6.12 | 7.2-8.1 | 21 |
| 4700 | 1980 | 5.73 | 8.8-9.7 | 39 |

The second table shows that compressibility and comparability were acceptable when direct tableting the multi-functional particulate system, since both thickness (i.e., tablet volume) and hardness were sensitive to compression force.

### Example 3

### Carrying Capacity of Multi-Functional Particulate Systems

The present example helped to determine the optimum carrying capacity of multi-functional particulate systems using acetaminophen.

Equipment: V-blender, instrumented Stokes RB-2 press; four 11/16″ flat-faced beveled-edged punches.
3a. Formulation (10% Acetaminophen):

$$\frac{\begin{matrix}42.0 g Acetaminophen \\ 374.0 g multi \text{-} functional particulate system \\ 4.4 g Mg \text{-} stearate\end{matrix}}{420.0 g TOTAL WEIGHT}$$

TABLE 3
Tablets made at about 1200 mg tablet weight with 10%
Acetaminophen, at different compressions

| | Tablet | Thickness | Hardness range | Ejection |
|---|---|---|---|---|
| Compression (lbs) | Weight (mg) | (mm) | (kP) | (lbs) |
| 1900 | 1200 | 3.98 | 1.5-1.8 | 3 |
| 3100 | 1200 | 3.83 | 2.7-3.2 | 12 |
| 4000 | 1190 | 3.74 | 3.9-4.2 | 20 |
| 5200 | 1220 | 3.62 | 4.7-7.0 | 25 |
| 7200 | 1220 | 3.60 | 5.5-7.3 | 34 |
| 8400 | 1200 | 3.53 | 2.9-5.8 | 31 |

The multi-functional particulate system with 10% acetaminophen showed excellent compactability, A plateau starts at 5000 lb compression, with breakdown taking place between 7000 and 8000 lb. The plateau is the point at which the hardness does not increase with added compression. The plateau begins at high compression indicating that the multi-functional particulate system can easily carry 10% acetaminophen.

3b. Formulation (20% Acetaminophen):

$$\frac{\begin{matrix}101.0 g Acetaminophen ( \sim 20 \% ) \\ 399.0 g multi \text{-} functional particulate system ( \sim 79 \% ) \\ 5.0 g Mg \text{-} stearate ( \sim 1 \% )\end{matrix}}{505.0 g TOTAL WEIGHT}$$

TABLE 4
Tablets made at 1200 mg tablet weight with 20%
Acetaminophen, at different compressions

| | Tablet | Thickness | Hardness range | Ejection |
|---|---|---|---|---|
| Compression (lbs) | Weight (mg) | (mm) | (kP) | (lbs) |
| 2000 | 1230 | 4.18 | 1.2-1.6 | 3 |
| 2800 | 1200 | 3.90 | 2.0-2.4 | 10 |
| 3900 | 1220 | 3.94 | 3.1-3.7 | 17 |
| 5000 | 1210 | 3.50 | 3.6-4.3 | 21 |
| 5900 | 1220 | 3.78 | 3.0-3.4 | 21 |
| 8000 | 1200 | 3.75 | 0.8-1.5 | 18 |

Acceptable compactability is found up to about 5000 lb compression, Above that compression, breakdown sets in. The multi-functional particulate system shows acceptable carrying capacity for 20% acetaminophen.

3c. Formulation (30% Acetaminophen):

$$\frac{\begin{matrix}200.0 g Acetaminophen ( \sim 30 \% ) \\ 460.0 g multi \text{-} functional particulate system ( \sim 69 \% ) \\ 6.8 g Mg \text{-} stearate ( \sim 1 \% )\end{matrix}}{666.8 g TOTAL WEIGHT}$$

TABLE 5
Tablets made at about 1200 mg tablet weight with 30%
Acetaminophen, at different compressions

| | Tablet | Thickness | Hardness range | Ejection |
|---|---|---|---|---|
| Compression (lbs) | Weight (mg) | (mm) | (kP) | (lbs) |
| 1500 | 1190 | 4.18 | 0.7-0.9 | 0 |
| 3100 | 1190 | 3.98 | 1.7-1.9 | 8 |
| 4100 | 1200 | 3.93 | 2.3-2.7 | 14 |
| 5000 | 1200 | 3.89 | 2.4-2.8 | 14 |
| 6000 | 1200 | 3.85 | 3.0-3.2 | 17 |
| 6600 | 1190 | 3.79 | 1.1-1.7 | 12 |

Compared with formulation 3b, (20% Acetaminophen), the plateau occurs at somewhat lower hardness, indicating that carrying capacity is at or near its limit.

3d. Formulation (40% Acetaminophen):

$$\frac{\begin{matrix}206.0 g Acetaminophen ( \sim 40 \% ) \\ 304.0 g multi \text{-} functional particulate systems ( \sim 59 \% ) \\ 5.2 g Mg \text{-} stearate ( \sim 1 \% )\end{matrix}}{515.2 g TOTAL WEIGHT}$$

TABLE 6
Tablets made at 1200 mg tablet weight with 40% Acetaminophen,
at different compressions

| | Tablet | Thickness | Hardness range | Ejection |
|---|---|---|---|---|
| Compression (lbs) | Weight (mg) | (mm) | (kP) | (lbs) |
| 2000 | 1210 | 4.27 | 0.9-1.0 | 0 |
| 3000 | 1220 | 4.14 | 1.4-1.6 | 6 |
| 4000 | 1220 | 4.08 | 1.7-2.1 | 11 |
| 5000 | 1210 | 4.01 | 2.0-2.4 | 13 |
| 6100 | 1200 | 3.92 | 1.2-4.6 | 10 |

The results indicate that multi-functional particulate system is not as effective at carrying acetaminophen at this level (40%).

### Example 4

### Enhancement of Multi-Functional Particulate System Carrying Capacity

ProSolv/S630 is a seven-to-one blend of silicified microcrystalline cellulose and vinyl-pyrollidone vinyl-acetate copolymer.

4a. Formulation (44% Acetaminophen with multi-functional particulate systems and ProSolv/S630):

$$\frac{\begin{matrix}84.0 g ProSolv / S 630 ( \sim 11 \% ) \\ 343.0 g Acetaminophen ( \sim 44 \% ) \\ 342.0 g multi \text{-} functional particulate system ( \sim 44 \% ) \\ 7.8 g Mg \text{-} stearate ( \sim 1 \% )\end{matrix}}{776.8 g TOTAL WEIGHT}$$

This experiment was performed to determine the carrying capacity of multi-functional particulate systems (see formulation 2d.). In addition to 44% Acetaminophen and the same amount of multi-functional particulate system, there was about 11% ProSolv/S630 in the compression mix.

TABLE 7
The compression profile of the Prosolv/S630 (7/1), 44%
Acetaminophen, and Multi-functional Particulate System mixture

| | Tablet | Thickness | Hardness range | Ejection |
|---|---|---|---|---|
| Compression (lbs) | Weight (mg) | (mm) | (kP) | (lbs) |
| 4000 | 1210 | 4.24 | 2.2-2.7 | 14 |
| 5100 | 1210 | 4.16 | 2.5-2.8 | 16 |
| 6000 | 1210 | 4.10 | 3.3-3.5 | 18 |
| 6500 | 1190 | 4.07 | 3.2-3.6 | 17 |
| 7000 | 1220 | 4.08 | 2.8-3.1 | 19 |
| 8200 | 1210 | 4.06 | 2.1-2.5 | 21 |

The Prosolv/S630 (7/1), 44% Acetaminophen, and Multi-functional Particulate System mixture shows a substantial improvement over formulation 2d. ProSolv/S630 works synergistically with the multi-functional particulate system to increase carrying capacity of Acetaminophen.

4b. Formulation (55% Acetaminophen with multi-functional particulate systems and ProSolv/S630):

$$\frac{\begin{matrix}40.0 g ProSolv / S 630 ( \sim 9 \% ) \\ 240.0 g Acetaminophen ( \sim 55 \% ) \\ 156.0 g Multi \text{-} functional Particulate System ( \sim 35 \% ) \\ 4.4 g Mg \text{-} stearate ( \sim 1 \% )\end{matrix}}{440.4 g TOTAL WEIGHT}$$

The relative amount of raw Acetaminophen was increased to further stress the carrying capacity of multi-functional particulate systems; ProSolv/S630 was kept at. about 9% of the tablet weight.

TABLE 8
The compression profile of the Prosolv/S630 (7/1), 55%
Acetaminophen, and muiti-functional particulate system mixture

| | Tablet | Thickness | Hardness range | Ejection |
|---|---|---|---|---|
| Compression (lbs) | Weight (mg) | (mm) | (kP) | (lbs) |
| 5000 | 1220 | 4.31 | 1.5-2.0 | 14 |
| 6000 | 1220 | 4.30 | 1.4-1.9 | 16 |
| 7000 | 1220 | 4.28 | 1.8-2.1 | 18 |
| 8500 | 1220 | 4.18 | 1.6-2.1 | 20 |
| 10000 | 1200 | 4.12 | 1.1-1.5 | 20 |

The hardness ranges indicate borderline acceptable tensile strength at best.

4c Formulations (high Acetaminophen, low multi-functional particulate systems and ProSolv/S630):

Additional formulations made at higher Acetaminophen (>60%) and increased ProSolv levels produced tablets with hardness below 2.0 kP, indicating that the carrying capacity of multi-functional particulate systems/ProSolv/S630 system is about 50% Acetaminophen, which is approximately 20% greater than the multi-functional particulate system alone.

### Example 5

### Cohesive Ingredient Dispersion with Multi-Functional; Particulate Systems

Ubidecarenone (ubiquinone; CoQ 10) does not flow due to its cohesive character. It also has content-uniformity issues in solid dosage forms. Therefore, it is extremely difficult to tablet by itself.

The multi-functional particulate system was used to disperse the fine particles of CoQ10and enhance flow. Colloidal silica (CabOSil) and high-density silicified microcrystalline cellulose (ProSolv HD90) were used as blending agents.

### Formulation:

$$\frac{\begin{matrix}44.0 \% Ubidecarenone \\ 1.3 \% CabOSil \\ 5.0 \% ProSolv HD 90 \\ 44.0 \% Multi \text{-} functional Particulate System \\ 4.4 \% Polyplasdone Crospovidone \\ 1.3 \% Mg \text{-} stearate\end{matrix}}{100.0 \% TOTAL}$$

### Protocol:

CabOSil with ubidecarenone is co-sieved through a 30-mesh. The ProSolv HD90 and multi-functional particulate system are added. The powder mix is blended at maximum speed for ten minutes. The powder mix is then sieved through a 40-mesh. Polyplasdone Crospovidone is added, and the mixture is blended at maximum speed for two minutes Finally, Mg-stearate is added, and the mixture is blended at maximum speed for two minutes.

The powder mixture showed remarkable improvement in flow. The bulk density of the final blend was 0.45 g/mL. The incorporation of a multi-functional particulate system with a substantially unflowable particle like CoQ10 allows it to flow.

### Example 6

### Incorporation of Encapsulated Pharmacological Agents

The present experiment was performed to determine applicability of encapsulated vitamin C (Vitashure™ 160 sold by Bale-hem Corporation) for making 500 mg active Vitamin C tablets, using multi-functional particulate systems as the main filler, and to measure the compression profile.

Equipment: V-blender; instrumented Stokes RB-2 press; four 11/16″ flat-faced beveled-edged punches.

### Formulation:

$$\frac{\begin{matrix}400.0 g Vitashure 160 ( 70 \% vitamin C active , Balchem Corporation ) \\ 400.0 g Multi \text{-} functional Particulate System \\ 100.0 g sorbitol \\ 20.0 g Starch 1500 \\ 20.0 g ProSolv SMCC 90 \\ 40.0 g Polyplasdone \\ 10.0 g sucralose \\ 10.0 g Mg \text{-} stearate\end{matrix}}{1000.0 g TOTAL WEIGHT}$$

TABLE 9
The compression profile of Vitamin C with a Muiti-functional
Particulate System

| | Tablet | Thickness | Hardness range | Ejection |
|---|---|---|---|---|
| Compression (lbs) | Weight (mg) | (mm) | (kP) | (lbs) |
| 2000 | 1810 | 6.09 | 5.1-5.5 | 5 |
| 2500 | 1810 | 5.99 | 6.7-7.0 | 8 |
| 3300 | 1830 | 5.86 | 9.7-10.1 | 11 |
| 5000 | 1810 | 5.60 | 12.1-12.7 | 19 |
| 7000 | 1830 | 5.52 | 16.4-16.8 | 25 |

Excellent tablets were made. The high tensile strength Indicates good compatibility between components, and also that sorbitol is a complementary excipient in this system.

Thus, while there have been described what are presently believed to be the preferred embodiments of the present invention, other and further embodiments and modifications can be made without departing from the spirit of this invention, and it is intended to include all such embodiments and modifications which come within the scope of the invention as set forth in the appended claims.

1. A pharmacologic dosage unit comprising:
(i) a pharmacologically active agent, and
(ii) a multi-functional particulate system.

2. A dosage unit according to claim 1 selected from the group consisting of tablets, sachets, lozenges, hard capsules, softgels, troches, dragees, suppositories, and the like

3. A dosage unit according to claim 2, which is a compressed tablet.

4. A dosage unit according to claim 1 wherein said multi-functional particulate system comprises: (i) solid biologically-safe particles, (ii) incorporated into a matrix, (iii) said incorporation having a characteristic that each individual particle retains its original size and said matrix being present in a weight ratio of 1:99 to 99:1, and (iv) wherein the mean weight diameter of resulting particulates have a size of from 25 to 500 microns.

5. A dosage unit according to claim 4 wherein said matrix comprises biologically-safe material selected from the group consisting of polysaccharides, modified polysaccharides, sugars, gums, thickeners, stabilizers, syrups, flours, sugar alcohols, starches, dextrose, maltodextrins, cellulose, and combinations thereof.

6. A dosage unit according to claim 5 wherein said matrix material is sugar or mixtures of sugars.

7. A dosage unit according to claim 1 wherein the multi-functional particulate system displays a loose hulk density of 0.1 to 1.1 kg/L.

8. A dosage unit according to claim 1 wherein the mean weight diameter of the multi-functional particulate system is from 50 to 400 microns.

9. A dosage unit according to claim 4 wherein said weight ratio between particles and matrix ranges from 20:80 to 80:20.

10. A dosage unit according to claim 4 wherein said weight ratio between particles and matrix ranges from 40:60 to 60:40.

11. A dosage unit according to claim 4 wherein said particles have a discrete particle size of 2 to 275 microns.

12. A dosage unit according to claim 4 wherein said particles are nutritionally active.

13. A dosage unit according to claim 4 wherein said particles comprise a material selected from the group consisting of Na₂CO₃, NaHCO₃, K₂CO₃, KHCO₃, CaCO₃, Ca(HCO₃₎₂, CaSO₄, Ca(NO₃)₂, CaSO₃, Ca(HSO₃)₂, MgCO₃, Mg(HCO₃)₂, Ca(HPO₄), Ca₃(PO₄)₂ citric acid, maleic acid, tartaric acid, maleic acid, lactic acid, acetic acid and combinations thereof.

14. A dosage unit according to claim 13 wherein said particle comprises CaCO₃.

15. A dosage unit according to claim 1 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, anti diarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants. antiobesity drugs, anabolic drugs, erythropoietic drugs, antiasthmatics, cough suppressants, mucolytics, anti-uricemic drugs, vitamins, and mixtures thereof,

16. A dosage unit according to claim 15 wherein said pharmacologically active agent is acetaminophen.

17. A dosage unit according to claim 15 wherein said pharmacologically active agent is Coenzyme Q10.

18. A dosage unit according to claim 15 wherein said pharmacologically active agent is encapsulated.

19. A method of preparing a pharmacologic dosage unit comprising:
admixing a multi-functional particulate system and a pharmacologically active agent in amounts requisite to provide a pharmacologic composition capable of use as an ingredient for inclusion in a pharmacologic dosage unit.

20. A method according to claim 19, wherein said dosage unit is selected from the group consisting of tablets, sachets, lozenges, hard capsules, softgels, troches, dragees, suppositories, and the like.

21. A method according to claim 20 wherein said pharmacologic composition has enhanced flowability.

22. A method according to claim 20 wherein said dosage unit is a compressed tablet.

23. A method according to claim 22 wherein said pharmacologic composition has enhanced comparability.

24. A method according to claim 19 wherein said multi-functional particulate system comprises: (i) solid biologically-safe particles, (ii) incorporated into a matrix, (iii) said incorporation having a characteristic that each individual particle retains its original size and said matrix being present in a weight ratio of 1:99 to 99:1, and (iv) wherein the mean weight diameter of resulting particulates have a size of from 25 to 500 microns.

25. A method according to claim 24 wherein said matrix comprises biologically-safe material selected from the group consisting of polysaccharides, modified polysaccharides, sugars, gums, thickeners, stabilizers, syrups, flours, sugar alcohols, starches, dextrose, maltodextrins, cellulose, and combinations thereof.

26. A method according to claim 25 wherein said matrix material is sugar or mixtures of sugars.

27. A method according to claim 19 wherein the multi-functional particulate system displays a loose bulk, density of 0.1 to 1.1 kg/L.

28. A method according to claim 19 wherein the mean weight diameter of the multi-functional particulate system is from 50 to 400 microns.

29. A dosage unit according to claim 24 wherein said weight ratio between particles and matrix ranges from 20.80 to 80:20.

30. A dosage unit according to claim 24 wherein said weight ratio between particles and matrix ranges from 40:60 to 60:40.

31. A method according to claim 24 wherein said particles have a discrete particle size of 2 to 275 microns.

32. A method according to claim 24 wherein said particles are nutritionally active.

33. A method according to claim 24 wherein said particles comprise a material selected from the group consisting of: Na₂CO₃, NaHCO₃, K₂CO₃, KHCO₃, CaCO₃, Ca(HCO₃₎₂, CaSO₄, Ca(NO₃)₂, CaSO₃, Ca(HSO₃)₂, MgCO₃, Mg(HCO₃)₂, Ca(HPO₄), Ca₃(PO₄)₂ citric acid, maleic acid, tartaric acid, maleic acid, lactic acid, acetic acid and combinations thereof.

34. A method according to claim 33 wherein said particle comprises CaCO₃.

35. The method according to claim 19 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, anti diarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic, agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

36. The method according to claim 35 wherein said pharmacologically active agent is acetaminophen.

37. The method according to claim 35 wherein said pharmacologically active agent is Coenzyme Q10.

38. The method according to claim 35 wherein said pharmacologically active agent is encapsulated.

39. A method for making a pharmacologic dosage unit comprising:
(i) admixing a multi-functional particulate system and a pharmacologically active agent in amounts requisite to provide a pharmacologic composition which is sufficiently flowable and compactable to form a compressed tablet; and
(ii) compacting the mixture resulting from step (i) sufficiently to form a compressed tablet.

40. A method according to claim 39, wherein said multi-functional particulate system comprises: (i) solid biologically-safe particles, (ii) incorporated into a matrix, (iii) said incorporation having a characteristic that each individual particle retains its original size and said matrix being present in a weight ratio of 1:99 to 99:1, and (iv) wherein the mean weight diameter of resulting particulates have a size of from 25 to 500 microns.

41. A method according to claim 40 wherein said matrix is biologically-safe and is selected from the group consisting of polysaccharides, modified polysaccharides, sugars, gum, thickeners, stabilizers, syrups, flours, starches, dextrose, maltodextrins, cellulose, and combinations thereof.

42. A method according to claim 39 wherein the multi-functional particulate system displays a loose bulk density of 0.1 to 1.1 kg/L.

43. A method according to claim 39 wherein the mean weight diameter of the multi-functional particulate system is from 50 to 400 microns.

44. A dosage unit according to claim 40 wherein said weight ratio between particles and matrix ranges from 20:80 to 80:20.

45. A dosage unit according to claim 40 wherein said weight ratio between particles and matrix ranges from 40:60 to 60:40.

46. A method according to claim 40 wherein said particles have a discrete particle size of 2 to 275 microns.

47. A method according to claim 40 wherein said particles are nutritionally active.

48. A method according to claim 40 wherein said particles are selected from the group consisting of: Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, citric acid, maleic acid, tartaric acid, maleic acid, lactic acid, acetic acid and derivatives and salts thereof.

49. The method according to claim 39 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, anti diarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

50. The method according to claim 49 wherein said pharmacologically active agent is acetaminophen.

51. The method according to claim 49 wherein said pharmacologically active agent is Coenzyme Q10.

52. The method according to claim 49 wherein said pharmacologically active agent is encapsulated,

53. A process for enhancing flow properties of a pharmacologic composition used as an ingredient for a pharmacologic dosage unit comprising:
adding a multi-functional particulate system to at least one pharmacologically active agent in an amount sufficient to improve flowability of said active agent in its use as an ingredient for making a pharmacologic dosage unit.

54. A process according to claim 53 wherein said dosage unit is selected from the group consisting of tablets, sachets, lozenges, hard capsules, softgels, troches, dragees, suppositories, and the like.

55. A process according to claim 54 wherein said dosage unit is a compressed tablet.

56. A process according to claim 53 wherein said multi-functional particulate system comprises: (i) solid biologically-safe particles, (ii) incorporated into a matrix, (iii) said incorporation having a characteristic that each individual particle retains its original size and said matrix being present, in a weight ratio of 1:99 to 99:1, and (iv) wherein the mean weight diameter of resulting particulates have a size of from 25 to 500 microns.

57. A process according to claim 56 wherein said matrix comprises biologically-safe material selected from the group consisting of polysaccharides, modified polysaccharides, sugars, gums, thickeners, stabilizers, syrups, flours, sugar alcohols, starches, dextrose, maltodextrins, cellulose, and combinations thereof.

58. A process according to claim 57 wherein said matrix material is sugar or mixtures of sugars.

59. A process according to claim 56 wherein said particles comprise a material selected from the group consisting of: Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, Ca(HPO₄), Ca₃(PO₄)₂ citric acid, maleic acid, tartaric acid, maleic acid, lactic acid, acetic acid and combinations thereof.

60. A process according to claim 59 wherein said particle comprises CaCO₃.

61. A process according to claim 53 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

62. A process according to claim 61 wherein said pharmacologically active agent is acetaminophen.

63. A process according to claim 61 wherein said pharmacologically active agent is Coenzyme Q10.

64. A process according to claim 61 wherein said pharmacologically active agent is encapsulated.

65. A process for enhancing compacting properties of a pharmacologic composition used as an ingredient for a pharmacologic dosage unit comprising:
adding a multi-functional particulate system to at least one pharmacologically active agent in an amount sufficient to provide or improve compactability of said active agent whereby it can be used as an ingredient for making a pharmacologic dosage unit.

66. A process according to claim 65 wherein said dosage unit is selected from the group consisting of tablets, sachets, lozenges, hard capsules, softgels, troches, dragees, suppositories, and the like.

67. A process according to claim 66 wherein said dosage unit is a compressed tablet.

68. A process according to claim 65 wherein said multi-functional particulate system comprises: (i) solid biologically-safe particles, (ii) incorporated into a matrix, (iii) said incorporation having a characteristic that each individual particle retains its original size and said matrix being present in a weight ratio of 1:99 to 99:1, and (iv) wherein the mean weight diameter of resulting particulates have a size of from 25 to 500 microns.

69. A process according to claim 68 wherein said matrix comprises biologically-safe material selected from the group consisting of polysaccharides, modified polysaccharides, sugars, gums, thickeners, stabilizers, syrups, flours, sugar alcohols, starches, dextrose, maltodextrins, cellulose, and combinations thereof.

70. A process according to claim 69 wherein said matrix material is sugar or mixtures of sugars.

71. A process according to claim 68 wherein said particles comprise a material selected from the group consisting of: Na₂CO₃, NaHCO₃. K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, Ca(HPO₄), Ca₃(PO₄)₂ citric acid, maleic acid, tartaric acid, maleic acid, lactic acid, acetic acid and combinations thereof.

72. A process according to claim 71 wherein said particle comprises CaCO₃.

73. A process according to claim 65 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, anti diarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

74. A process according to claim 73 wherein said pharmacologically active agent is acetaminophen.

75. A process according to claim 73 wherein said pharmacologically active agent is Coenzyme Q10.

76. A process according to claim 73 wherein said pharmacologically active agent is encapsulated.

77. A method for delivering a pharmacologically active agent to a patient, comprising:
administering to a patient, in need of treatment with at least one pharmacologically-active agent, a pharmacologic dosage unit, comprising:
(i) at least one pharmacologically active agent suitable for said treatment of said patient, and
(ii) a multi-functional particulate system.

78. A method according to claim 77 wherein said dosage unit is selected from the group consisting of tablets, sachets, lozenges, hard capsules, softgels, troches, dragees, suppositories, and the like.

79. A method according to claim 78 wherein said dosage unit is a compressed tablet.

80. A method according to claim 77 wherein said multi-functional particulate system comprises: (i) solid biologically-safe particles, (ii) incorporated into a matrix, (iii) said incorporation having a characteristic that each individual particle retains its original size and said matrix being present in a weight ratio of 1:99 to 99:1, and (iv) wherein the mean weight diameter of resulting particulates have a size of from 25 to 500 microns.

81. A method according to claim 80 wherein said matrix comprises biologically-safe material selected from the group consisting of polysaccharides, modified polysaccharides, sugars, gums, thickeners, stabilizers, syrups, flours, sugar alcohols, starches, dextrose, maltodextrins, cellulose, and combinations thereof.

82. A method according to claim 81 wherein said matrix material is sugar or mixtures of sugars.

83. A method according to claim 80 wherein said particles comprise a material selected from the group consisting of Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, Ca(HPO₄), Ca₃(PO₄)₂ citric acid, maleic acid, tartaric acid, maleic acid, lactic acid, acetic acid and combinations thereof.

84. A method according to claim 83 wherein said particle comprises CaCO₃.

85. A method according to claim 77 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

86. A method according to claim 85 wherein said pharmacologically active agent is acetaminophen.

87. A method according to claim 85 wherein said pharmacologically active agent is Coenzyme Q10.

88. A method according to claim 85 wherein said pharmacologically active agent is encapsulated.

89. A method for increasing capacity for inclusion of at least one ingredient in a pharmacologic dosage unit comprising:
adding a multi-functional particulate system to a pharmacologic composition used in a pharmacologic dosage unit in an amount sufficient to increase the amount of at least one other ingredient in said composition.

90. A method according to claim 89 wherein said dosage unit is selected from the group consisting of tablets, sachets, lozenges, hard capsules, softgels, troches, dragees, suppositories, and the like.

91. A method according to claim 90 wherein said dosage unit is a compressed tablet.

92. A method according to claim 89 wherein said multi-functional particulate system comprises: (i) solid biologically-safe particles, (ii) incorporated into a matrix, (iii) said incorporation having a characteristic that each individual particle retains its original size and said matrix being present in a w;eight ratio of 1:99 to 99:1, and (iv) wherein the mean weight diameter of resulting particulates have a size of from 25 to 500 microns.

93. A method according to claim 92 wherein said matrix comprises biologically-safe material selected from the group consisting of polysaccharides, modified polysaccharides, sugars, gums, thickeners, stabilizers, syrups, flours, sugar alcohols, starches, dextrose, maltodextrins, cellulose, and combinations thereof.

94. A method according to claim 93 wherein said matrix material is sugar or mixtures of sugars.

95. A method according to claim 92 wherein said particles comprise a material selected from the group consisting of: Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, Ca(HPO₄), Ca₃(PO₄)₂ citric acid, maleic acid, tartaric acid, maleic acid, lactic acid, acetic acid and combinations thereof.

96. A method according to claim 95 wherein said particle comprises CaCO₃.

97. A method according to claim 89 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral, psychotropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants., antiobesity drugs, anabolic drugs, erythropoietic drugs, antiasthmatics, cough suppressants, mucolytics, anti-uricemic drugs, vitamins, and mixtures thereof.

98. A method according to claim 97 wherein said pharmacologically active agent is acetaminophen.

99. A method according to claim 97 wherein said pharmacologically active agent is Coenzyme Q10.

100. A method according to claim 97 wherein said pharmacologically active agent, is encapsulated.

101. A method according to claim 89 wherein said pharmacologic composition comprises an excipient selected from the group consisting of silicified microcrystalline cellulose, vinyl-pyrollidone vinyl-acetate copolymer, or combinations thereof.

102. A method for increasing dispersion of pharmacologically active agents within a pharmacologic dosage unit comprising:
adding a multi-functional particulate system to at least one pharmacologically active agent in an amount sufficient to provide or improve dispersion of said pharmacologically active agent whereby its inclusion as an ingredient in a pharmacologic dosage unit is facilitated.

103. A method according to claim 102 wherein said dosage unit is selected from the group consisting of tablets, sachets, lozenges, hard capsules, softgels, troches, dragees, suppositories, and the like.

104. A method according to claim 103 wherein said dosage unit is a compressed tablet.

105. A method according to claim 102 wherein said multi-functional particulate system comprises: (i) solid biologically-safe particles, (ii) incorporated into a matrix, (iii) said incorporation having a characteristic that each individual particle retains its original size and said matrix being present in a weight ratio of 1:99 to 99:1, and (iv) wherein the mean weight diameter of resulting particulates have a size of from 25 to 500 microns,

106. A method according to claim 105 wherein said matrix comprises biologically-safe material selected from the group consisting of polysaccharides, modified polysaccharides. sugars, gums, thickeners, stabilizers, syrups, flours, sugar alcohols, starches, dextrose, maltodextrins, cellulose, and combinations thereof.

107. A method according to claim 106 wherein said matrix material is sugar or mixtures of sugars.

108. A method according to claim 105 wherein said particles comprise a material selected from the group consisting of: Na₂CO₃, NaHCO₃, K₂CO₃, KHCO₃, CaCO₃, Ca(HCO₃)₂, CaSO₄, Ca(NO₃)₂, CaSO₃, Ca(HSO₃)₂, MgCO₃, Mg(HCO₃)₂, Ca(HPO₄), Ca₃(PO₄₎₂ citric acid, maleic acid, tartaric acid, maleic acid, lactic acid, acetic acid and combinations thereof.

109. A method according to claim 108 wherein said particle comprises CaCO₃.

110. A method according to claim 102 wherein said pharmacologically active agent is selected from the group consisting of: antitussives, antihistamines, decongestants, alkaloids, laxatives, ion-exchange resins, anti-cholesterolemic, anti-lipid agents, antiarrhythmics, antipyretics, analgesics, appetite suppressants expectorants, anti-anxiety agents, anti-ulcer agents, anti-inflammatory substances, coronary dilators, cerebral psychotropics, antimanics, stimulants, gastrointestinal agents, sedatives, antidiarrheal preparations, anti-anginal drugs, vasodialators, anti-hypertensive drugs, vasoconstrictors, migraine treatments, antibiotics, tranquilizers, antipsychotics, antitumor drugs, anticoagulants, antithrombotic drugs, hypnotics, anti-emetics, anti-nauseants, anticonvulsants, neuromuscular drugs, hyper- and hypoglycemic agents, thyroid and anti-thyroid preparation, diuretics, antispasmodics, uterine relaxants, antiobesity drugs, anabolic drugs, erythropoietic drugs, antiasthmatics, cough suppressants, mucolytics, anti-uricemic drugs, vitamins, and mixtures thereof.

111. A method according to claim 110 wherein said pharmacologically active agent is acetaminophen.

112. A method according to claim 110 wherein said pharmacologically active agent is Coenzyme Q10.

113. A method according to claim 110 wherein said pharmacologically active agent is encapsulated.
